(19) United States
(12) Patent Application Publication
(10) Pub. No.: US 2021/0251562 A1
(43) Pub. Date: Aug. 19, 2021

(54) SYSTEM AND DEVICE FOR DIAGNOSING AND MANAGING ERECTILE DYSFUNCTION

(71) Applicant: Abhinav JAIN, Agra (IN)

(72) Inventors: Abhinav JAIN, Agra (IN); Timo TSCHARNTKE, Laatzen (DE)

(21) Appl. No.: 16/973,788

(22) PCT Filed: Jun. 10, 2019
(86) PCT No.: PCT/IB2019/054801

§ 371 Date: Dec. 10, 2020

Related U.S. Application Data
(60) Provisional application No. 62/827,945, filed on Apr. 2, 2019.
(60) Provisional application No. 62/683,088, filed on Jun. 11, 2018.

(51) Int. Cl. A61B 5/00; A61B 5/0205; G16H 40/63; G16H 20/40

(52) U.S. Cl. CPC A61B 5/4393; A61B 5/6803; A61B 5/4812; A61B 5/02438; A61B 5/7282; G16H 40/63; G16H 20/40; A61B 5/0205

(57) ABSTRACT

A system for monitoring erectile dysfunction of a user includes a penile device configured to be accommodated on penis to evaluate penile tumescence, rigidity, and bio-impedance of penile shaft of the penis; and a head-band device implemented as a sleeping mask with electrodes strategically placed around the eyes of the user for detecting sleep characteristics, electrooculography (EOG), and electroencephalography (EEG). A computing device is operatively coupled to penile device and the head-band device to detect condition of rapid eye movement (REM) sleep phase and a condition of penile tumescence, and to actuate the penile devise to measure rigidity of the penile shaft to qualify condition of the erectile dysfunction as organic or inorganic.

### TECHNICAL FIELD

The present disclosure generally relates to the field of diagnosing the medical condition of erectile dysfunction (ED) and, more particularly, to a system and device for monitoring, diagnosing, distinguishing, and managing Erectile Dysfunction (ED).

### BACKGROUND

Erectile dysfunction (ED) is a medical condition characterized by the inability to achieve or sustain a penile erection necessary for sexual intercourse. The causes of Erectile Dysfunction are generally categorized as organic, psychogenic or a mix of both. Organic ED is a condition which arises from physiological issues with the patient's reproductive system. Examples of these issues are trauma, hormonal-imbalance and clogged arteries causing insufficient blood-flow. Psychogenic ED is caused by psychological issues such as stress, low self-esteem, fear and depression. In some rare cases, ED may arise due to neurological issues as well.

Established clinical tests and protocols lack in initial diagnosis phases in determining the type of ED affecting the patient. Specifically, the existing clinical best-practices for ED diagnosis, both in Europe and USA and even across the world, rely on general physical examination and computed score from a questionnaire as primary screening steps to assess likelihood of erectile dysfunction. The tests are performed by urologists, who are specifically-trained and experienced to handle ED cases. Diagnoses of ED cases during initial medical assessments and consultations may be inconclusive as there are different causes of ED for different patients. Identifying the exact cause of ED can result in more consultation sessions and tests, increasing the overall cost, and delaying the overall process. These delays and cost can further aggravate patients' psychological and economic condition.

Further, conventional solutions provide for treatment of all variations of erectile dysfunction through drugs. In addition to drug treatment, other approaches have been developed to treat ED, including for example, hormone therapy and surgery, including vascular reconstructive surgery.

The conventional solutions, such as questionnaires, are not objective and do not commonly take into account various physiological factors, for example, Nocturnal penile tumescence (NPT). Nocturnal penile tumescence is a spontaneous erection of the penis during rapid-eye movement (REM) sleep and is an objective parameter for accessing the nature of erectile dysfunction. Furthermore, existing methods for NPT measurements may give inconclusive or false-positive results in patients who suffer from other health issues such as sleep disorders, as sleep disorders affects NPT. In addition, products associated with conventional approach are cumbersome, complex to operate and may require presence of a trained-operator, especially when used at an in-patient clinic with sleep monitoring system. While prescribing an ED drug without reliable differential analysis of ED is a prevalent practice, it involves a certain degree of risk and health consequences. Thus, it would be advantageous to have a viable option for accurate diagnosis of ED that is also simple and follows patient-centric approach.

The present invention avoids the problems/disadvantages noted above and overcomes other problems encountered in conventional methods. The objects, advantages and novel features of the invention may be realized and obtained by means of the instrumentalities and combinations particularly pointed out in the detailed description.

### SUMMARY

The present disclosure generally relates to the field of diagnosing the medical condition of erectile dysfunction (ED) and, more particularly, to a system and device for measuring and monitoring different bodily parameters which may also be used for monitoring, diagnosing, distinguishing, and managing Erectile Dysfunction (ED).

According to an aspect of the present disclosure, the proposed system for monitoring, diagnosing, and managing a condition of erectile dysfunction of a user includes a penile device configured to be accommodated on the penis of the user, wherein the penile device is configured to monitor and measure at least one of a penile tumescence, a rigidity, and a bio-impedance of penile shaft of the penis; and a head-band device configured to be accommodated on head of the user. The head-band device is implemented as a sleeping mask with electrodes strategically placed around the eyes of the user for detecting at least one of sleep characteristics: electrooculography (EOG), orelectroencephalography (EEG). In an aspect, the system further includes a computing device with the penile device and the head-band device operatively coupled to the computing device. The computing device includes a processor and a memory coupled to the processor, and gathers data on at least one of the penile tumescence, the rigidity, and the bio-impedance of the penile shaft of the penis of the user from the penile device, and at least one of the sleep characteristics, the electrooculography (EOG), and the electroencephalography (EEG) of the user from the head device. The computing device further processes the gathered data to qualify condition of the erectile dysfunction (ED) of the user.

In an aspect, the detection of the sleep characteristics by the head device may include detection of a REM sleep phase, and the computing device, on detection of the REM sleep phase and a condition of penile tumescence, actuates the penile device to measure the rigidity of the penile shaft, one or both of axial rigidity or radial rigidity, to qualify condition of the erectile dysfunction as organic or inorganic.

In an aspect, the head-band device may also determine heart-rate of the user from the electrooculography and the electroencephalography signals, and use the determined heart rate to determine sleep state of the user.

In an embodiment, the penile device is adjustable in size and accommodates a minimum penile circumference corresponding to a flaccid penis, and a maximum penile circumference corresponding to an erect penis, catering to variations in the flaccid and erect conditions from person to person.

In an embodiment, the penile device is adapted to be accommodated around the penile shaft, and includes an elastomeric band; and an elastomeric housing. The housing includes an electronic circuitry to measure bio-impedance through a bio-impedance sensor. It further includes a tumescence and rigidity sensor for measuring both tumescence and rigidity. In an embodiment, the tumescence and rigidity sensor can include a first element for measuring strain to detect tumescence, and a second element to perform linear actuation to apply a force for measuring radial rigidity. The radial rigidity is measured by measuring additional strain, which is generated as a result of the applied force, using the first element.

In an embodiment, the second element can be made of a shape memory alloy and may be configured in series with the first element, so as to stretch as a result of tumescence. The second element regains its original shape on activation by heat or applied electric current or any other type of signal, which results in application of additional radial force on the penile shaft.

In an embodiment, the penile device can be based on a sensor made of an electroactive polymer and arranged around circumference of the penile shaft. The sensor can be used to measure strain as a result of change in circumference to measure tumescence, as well as to alter an applied force on the circumference of the penile shaft for measuring rigidity.

In an embodiment, the penile device may also include an inward extending force sensor to estimate a cavernosal pressure of the penis shaft. The cavernosal pressure and its periodic variations can be used to estimate the rigidity.

In an embodiment, the penile device can include a plurality of touch sensors provided on the elastic band on either side of a protruding notch. The touch sensors can provide a means to measure penile rigidity on coming in contact with the penile skin based upon respective erect or flaccid state of the penis.

In an embodiment, the penile device can be implemented as a plurality of the penile devices located along length of the penile shaft, which can be coupled to each other through one or more length-adjustable connectors.

In an alternate embodiment, the penile device can include a two clamping plates placed on the penile shaft in a manner such that the penis is in between the two plates. The plates can be coupled to each other by at least one length-adjustable connector.

In an alternate embodiment, the penile device can be implemented as a device to measure an axial rigidity of the penis and can comprise at least one stretch sensor strap and at least two shape memory straps made of material able to change their shape back to a predetermined shape when subjected to an external activation signal. The stretch sensor strap and the shape memory straps are configured along length of the penile shaft. One of the at least two shape memory straps tries to bend the penis and the other of the at least two shape memory straps bends it back to a straight position. Therefore, one of the at least two shape-memory straps has its activated shape at an angle, whereas the other of the at least two shape-memory straps returns to a straight strap when activated.

In yet another embodiment, the penile device can be implemented as an iris diaphragm having number of leaves arranged to provide a quasi-circular aperture of variable size. The penile device can be arranged on the penis such that the leaves rest against the penile shaft with a constant gentle torque acting on the iris diaphragm. The iris diaphragm can have position tracking capability to detect change of circumference of the penile shaft to detect and measure tumescence. The iris diaphragm can also be used to determine radial rigidity by varying the torque which applies a radial force on the penile shaft, and determining the change in the circumference of the penile shaft corresponding to an applied torque.

In an embodiment, the disclosed system can have an artificial intelligence (AI) based machine learning module that enables differential diagnosis with higher accuracy by accounting for biological and racial variations.

In an embodiment, the system can comprise an interface configured to provide the user any or a combination of a graphical representation of various evaluated parameters, and a questionnaire test. Before the information displayed on the interface being accessible to the user, the system can confirm authenticity of the user based upon a unique code generated at the end of a test conducted on the user.

An aspect of the present disclosure relates to a penile device that has capability to measure penile tumescence as well as radial rigidity of penile shaft of a penis of a user. The penile device is adapted to be accommodated circumferentially around the penis shaft and includes an electronics housing and a stretch sensor. One end of the stretch sensor is physically and communicably coupled with the electronics housing. A gap filler is fastened between the electronics housing and the other end of the stretch sensor such that the electronics housing, the stretch sensor, and the gap filler take a flexible band shape for being accommodated on the penile shaft of the penis. The gap filler is of adjustable length to enable snug fitment of the band shaped penile device circumferentially around the penile shaft in a flaccid state of the penis.

In an aspect, the stretch sensor is stretchable, and stretches under a force exerted by the penis during erection from a flaccid state, and returns to its original shape when the penis returns to the flaccid state. Further, the stretch sensor, on being stretched, is adapted to provide a signal indicative of force exerted on the stretch sensor to detect and measure a penile tumescence.

In an aspect, the stretch sensor is physically coupled to the electronics housing through a linear actuator that can contract to reduce circumferential length of the penile device, reduction in circumferential length results in exerting a radial force on the penile shaft to measure axial rigidity of the penile shaft. The measurement of the axial rigidity is taken by measuring force exerted on the stretch sensor as a result of reduction in circumferential length of the penile device.

In an aspect, the linear actuator can be one or more of micro springs made of a shape memory alloy. The micro springs can be arranged between the stretch sensor and the electronics housing such that the micro springs stretch along with stretching of the stretch sensor during erection of the penis, or before, such as at the time of arranging the penile device around the penile shaft. The micro springs return to their original shape on activation by heat or electric current, or any other type of signal, which results in reduction in the circumferential length of the penile device.

In an aspect, the penile device can further include a force sensor disposed on the electronics housing. The force sensor protrudes towards the penile shaft to estimate a cavernosal pressure of the penis shaft. The cavernosal pressure and its periodic variation can be used to estimate the rigidity. The force sensor can have a width narrower than the stretch sensor, and can be mounted on a retractable mechanical system to make it project when force measurement is to be taken.

In an aspect, the penile device can further include means of identifying the length of the adjustable gap-filler. This may be achieved, for instance, using a linear encoder based on but not limited to capacitive, optical, inductive or magnetic technology that is integrated in the gap filler. In this aspect, the electronics housing has an electronic reader for the encoder. Combined with the dynamic stretch sensor measurement and the known length of the electronics housing, the signal from this linear encoder then allows for accurate determination of the penile circumference in absolute values.

Yet another aspect of the present disclosure relates to a method for monitoring, diagnosing, and managing a condition of erectile dysfunction of a user. The method comprises the steps of: (i) monitoring, using a penile device accommodated on penile shaft of penis of the user, at least one of a penile tumescence, a rigidity of a penile shaft of the penis, and a bio-impedance of the penile shaft; (ii) monitoring, using a head-band device configured on head of the user, at least one of sleep characteristics, electrooculography (EOG), and electroencephalography (EEG); (iii) detecting, based on monitoring of at least one of the sleep characteristics, the electrooculography (EOG), and the electroencephalography (EEG), if the user is in an REM state; (iv) detecting, when the user is in an REM state, based on monitoring of the penile tumescence, if the user in a condition of penile tumescence; (v) actuating, using a computing device operatively coupled to the penile device and the head-band device, when the user is in a condition of penile tumescence, the penile devise to measure the rigidity of the penile shaft; and (vi) qualifying, based on the measured rigidity of the penile shaft, condition of the erectile dysfunction as organic or inorganic.

Various objects, features, aspects and advantages of the present disclosure will become more apparent from the following detailed description of preferred embodiments, along with the accompanying drawing figures in which like numerals represent like features.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

FIG. 1A illustrates a system diagram for the proposed system for monitoring, diagnosing, and managing a condition of erectile dysfunction, in accordance with an embodiment of the present disclosure.

FIG. 1B illustrates a schematic representation of environment for implementing the disclosed system for monitoring, diagnosing, and managing a condition of erectile dysfunction, in accordance with an embodiment of the present disclosure.

FIG. 2A illustrates a penile device configured to be accommodated circumferentially around the penis, in accordance with an embodiment of the present disclosure.

FIG. 2B illustrates a penile circumference diagram and corresponding difference in its circumference between a flaccid state and erect state.

FIGS. 3A-3C illustrate different perspective views of the penile device of FIG. 2A showing the device adjusted to different sizes, in accordance with an embodiment of the present disclosure.

FIGS. 4A and 4B illustrate a two-dimensional representation of the penile device of FIG. 2A showing fitment of gap filler, in accordance with an embodiment of the present disclosure.

FIG. 5A illustrates the head-band device, in accordance with an embodiment of the present disclosure.

FIG. 5B illustrates exemplary locations of different electrodes when the head-band device of FIG. 5A is positioned on head of a user.

FIGS. 6A and 6B illustrate an exemplary screen shot of the user interface associated with the disclosed system, in accordance with an embodiment of the present disclosure.

FIGS. 7A and 7B illustrate different perspective views of a penile device 700, in accordance with another embodiment of the present disclosure.

FIG. 7C shows fitment of two penile devices on a penile shaft, in accordance with another embodiment of the present disclosure.

FIGS. 8A and 8B illustrate internal structure of the penile device, in accordance with another embodiment of the present disclosure.

FIGS. 9A and 9B illustrate coupling of stretch sensor and linear actuators of the penile device, in accordance with an embodiment of the present disclosure.

FIGS. 10A and 10B show two possible configurations of gap filler for adjusting circumferential length of the penile device, in accordance with another embodiment of the present disclosure.

FIGS. 11A-10D illustrate a penile device provided with length-adjustable gap fillers of different configurations, in accordance with different embodiments of the present disclosure.

FIG. 12 illustrates another exemplary embodiment implementing the system with two penile devices coupled by a length-adjustable connector, in accordance with an embodiment of the present disclosure.

FIG. 13 illustrates an exploded view showing coupling of the length-adjustable connector 1202 with the penile device, in accordance with an embodiment of the present disclosure.

FIG. 14 illustrates another configuration of the penile device, in accordance with an embodiment of the present disclosure.

FIG. 15 illustrates a penile device configured for measuring axial rigidity of the penile shaft, in accordance with another embodiment of the present disclosure.

FIG. 16A illustrates a penile device having a force sensor, in accordance with another embodiment of the present disclosure.

FIG. 16B illustrates geometry of the force-sensor of FIG. 16A, and its contact area, in accordance with another embodiment of the present disclosure.

FIG. 17 illustrates the mounting of a force sensor mounted on a retractable mechanical system, penile device in accordance with another embodiment of the present disclosure.

FIGS. 18A and 18B illustrate a penile device having touch sensors, in accordance with another embodiment of the present disclosure.

FIG. 19 illustrates an iris diaphragm based penile device, in accordance with another embodiment of the present disclosure.

FIG. 20 is a method flow diagram for the proposed method for monitoring, diagnosing, and managing a condition of erectile dysfunction, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

The present disclosure generally relates to the field of diagnosing the medical condition of erectile dysfunction (ED) and, more particularly, to a system and device for monitoring, diagnosing, distinguishing, and managing erectile dysfunction (ED).

The present invention provides a system, device, and a method for diagnosing, distinguishing, and managing erectile dysfunction (ED) of different types. ED is a medical condition characterized by the inability of a male patient to achieve or sustain a penile erection for sexual intercourse. The present invention distinguishes the condition of organic ED from psychogenic ED, a mix of both, or ED of other nature. The data, including patient's historical results from physical examination or questionnaires, is also processed by the system of the present invention to identify the patient's condition as organic ED, psychogenic ED, mix of both, or erectile disorders due to other causes. The invention identifies the underlying medical condition as hypogonadism, venous leakage or arterial blood-flow for qualifying organic ED. The diagnosis is made possible by an analysis of data obtained from the ED patient's bodily functions. The bodily data acquired by the system are one or more of penile tumescence, penile rigidity (axial or radial or both), penile electro-bio-impedance, motion-detection (actigraphy), detection of sleep-phase (including REM sleep) using one or more of actigraphy, electrooculography (EOG) and electroencephalography (EEG). Some of these terminologies are explained in the following description.

Rapid Eye Motion (REM) sleep is a phase of sleep in which the eyes of the person perform rapid and random movements. This sleep phase also accompanies low muscular tension throughout the body.

Nocturnal Penile Tumescence (NPT) is an involuntary erection of the penile shaft of a healthy male during REM sleep. This phenomenon is of high clinical value in understanding the nature of erectile dysfunction. Lack of NPT during nocturnal sleep has been attributed to presence of organic ED. In contrast, patients suffering from psychogenic ED and achieving REM sleep have a healthy tumescence with good rigidity. Thus, the presence of NPT can be used to distinguish among different kinds of ED. electrooculography (EOG) is the measurement of potential difference between cornea and retina of an eye and can be used for non-invasively detecting eye-movements during REM sleep. electroencephalography (EEG) is a method to record electrical activity of the brain of a patient. Actigraphy is a technique to determine human activities (including resting state).

Electro Bio-impedance is a measure of opposition to electric current when a voltage is applied across a biological tissue. It is frequently used for estimation of body fat and composition in human body. It can be used also for measuring blood flow across an organ or vessel. The ability to measure blood-flow provides a non-invasive way for real-time monitoring of blood in a tissue or organ.

The present invention also offers post-diagnostic assistance to the patient. Specifically, it provides an indication for the right ED treatment and management options to be undertaken by the patient. It is helpful to patients suffering from any type of ED, for example psychogenic ED, or organic ED. It has been engineered to keep privacy, convenience and ease-of-use as priorities to ensure reliable home-use by untrained patients or users. While the present invention is primarily designed for use in ED diagnosis and identification, the present invention can also be used for other medical purposes (such as for monitoring efficacy of a medicine, which may influence penile tumescence or rigidity, or nocturnal monitoring of sleep, psychological or cardiovascular disorders, to name a few) as well as non-medical purposes (such as lifestyle tracking of penile parameters during sexual intercourse or self-stimulation).

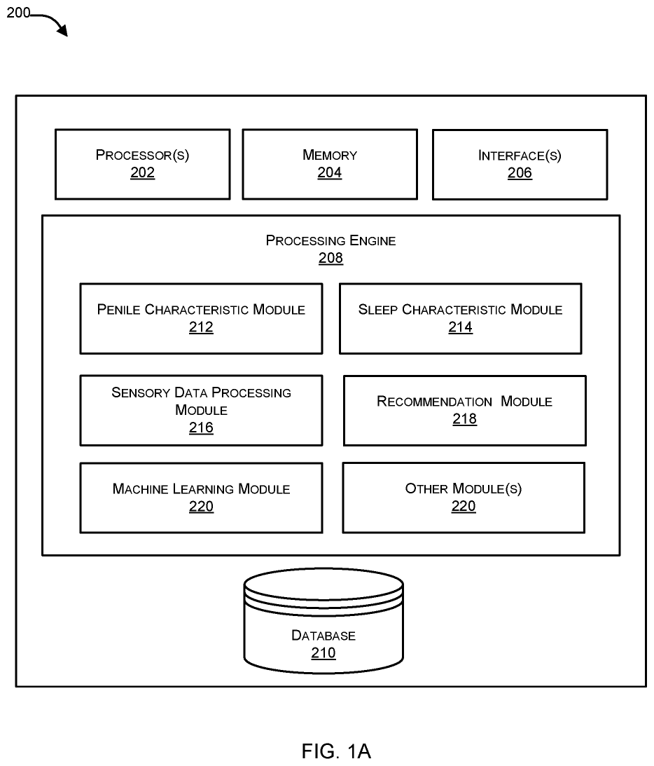

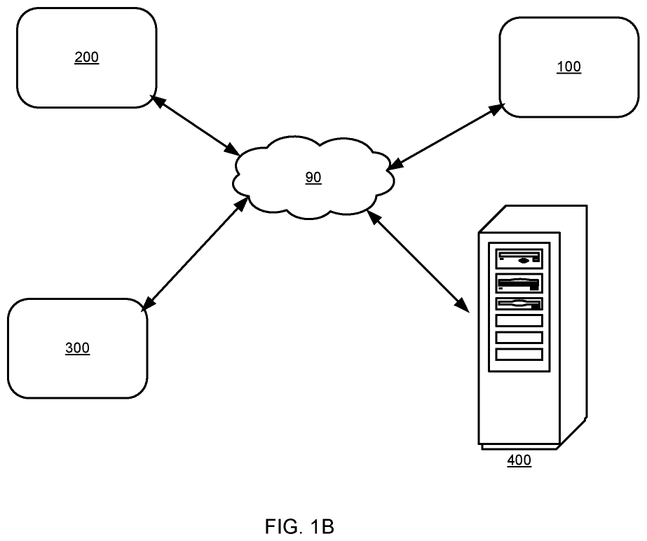

FIGS. 1A and 1B respectively illustrate a system diagram for the proposed system 200 for monitoring, diagnosing, and managing a condition of erectile dysfunction, and a schematic representation showing environment in which the system 200 works. In an embodiment, the system 200 is configured with a human body, and more particularly associated with a male body.

In an aspect, the system 200 may comprise a computing device having one or more hardware processor(s) 202. The one or more hardware processor(s) 202 may be implemented as one or more microprocessors, microcomputers, microcontrollers, digital signal processors, central processing units, logic circuitries, and/or any devices that manipulate data based on operational instructions. Among other capabilities, the one or more hardware processor(s) 202 are configured to fetch and execute computer-readable instructions stored in a memory 204 of the computing device. The memory 204 may store one or more computer-readable instructions or routines, which may be fetched and executed to create or share the data units over a network service. The memory 204 may comprise any non-transitory storage device including, for example, volatile memory such as RAM, or non-volatile memory such as EPROM, flash memory, and the like.

The system 200 also includes an interface(s) 206. The interface(s) 206 may comprise a variety of interfaces, for example, interfaces for data input and output devices, referred to as I/O devices, storage devices, and the like. The interface(s) 206 may facilitate communication of the system 200 with various devices coupled to the system 200 such as a penile device 100, a head-band device 300, and a server 400. In an embodiment, the system 200 may be communicably coupled to the penile device 100, the head-band device 300, and the server 400 over a network 90. In an example, the network 90 may be any wired or wireless network known to a person having ordinary skill in the art. The interface(s) 206 may also provide a communication pathway for one or more components of system 200. Examples of such components include, but are not limited to, processing engine(s) 208 and data 210.

The processing engine(s) 208 may be implemented as a combination of hardware and programming (for example, programmable instructions) to implement one or more functionalities of the processing engine(s) 208. In examples described herein, such combinations of hardware and programming may be implemented in several different ways. For example, the programming for the processing engine(s) 208 may be processor executable instructions stored on a non-transitory machine-readable storage medium and the hardware for the processing engine(s) 208 may comprise a processing resource (for example, one or more processors), to execute such instructions. In the present examples, the machine-readable storage medium may store instructions that, when executed by the processing resource, implement the processing engine(s) 208. In such examples, the system 200 may comprise the machine-readable storage medium storing the instructions and the processing resource to execute the instructions, or the machine-readable storage medium may be separate but accessible to system 200 and the processing resource. In other examples, the processing engine(s) 208 may be implemented by electronic circuitry.

The data 210 may comprise data that is either stored or generated as a result of functionalities implemented by any of the components of the processing engine(s) 208.

In an exemplary embodiment, the processing engine(s) 208 may comprise a penile characteristic module 212, a sleep characteristic module 214, a sensory data processing module 216, a recommendation module 218, machine learning module 220, and other module 222.

In an embodiment, the penile characteristic module 212 is implemented with a penile device, wherein the penile device is configured to monitor and measure penile tumescence, rigidity, and bio-impedance of penile shaft of the penis. Tumescence is defined as change of circumference (during an erection), and penile rigidity can be measured axially (resistance to bending) or radially (resistance to squeezing).

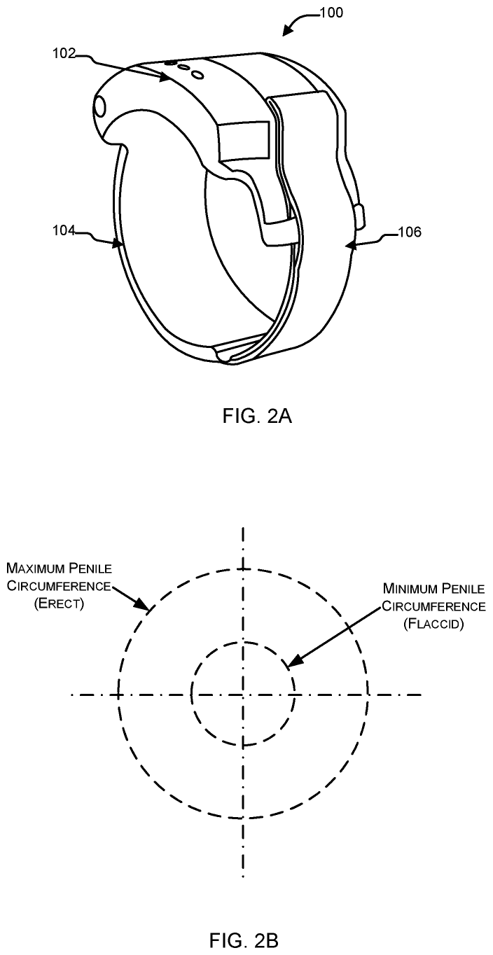

FIG. 2A illustrates an exemplary penile device 100 configured to be accommodated circumferentially around a penis in accordance with an embodiment of the present disclosure. In an example, a subject or patient places the penile device 100 on his penile shaft before going to bed at night or before a sleeping schedule. In an example, the penile device 100 is adjustable in size so as to accommodate all possible penile circumferences. Furthermore, the penile device 100 is able to accommodate changes in penile circumference during an erection. For example, the penile device 100 may accommodate a minimum penile circumference corresponding to a flaccid penis, and a maximum penile circumference corresponding to an erect penis, simultaneously catering to variations in penile circumference from person to person.

FIG. 2B illustrates a penile circumference diagram and corresponding difference between flaccid and erect penis circumference. In an example, the smaller inner circle corresponds to a flaccid state of the penis, the larger outer circle corresponds to an erect state of the penis. Human penis has tissues and blood vessels, which enable the penis to engorge during sexual excitation. The engorgement (also called tumescence) may vary among population and is a factor or genetic makeup and other physiological parameters such as blood flow and sensitivity of tissues towards nitrous oxide. These factors influence the size-distribution of the human penis with difference in change in circumferences for smallest flaccid penis to largest engorged penis. The present invention overcomes challenges in development variable size device, and provides a passive system for measuring penile erection to account for large variations.

Referring back to FIG. 2A, the penile device 100 includes an electronics housing 102, a stretch sensor 104, and a gap filler 106. The electronics housing 102, the stretch sensor 104, and the gap filler 106 together provide a flexible band shaped profile suitable for fitment on the penile shaft.

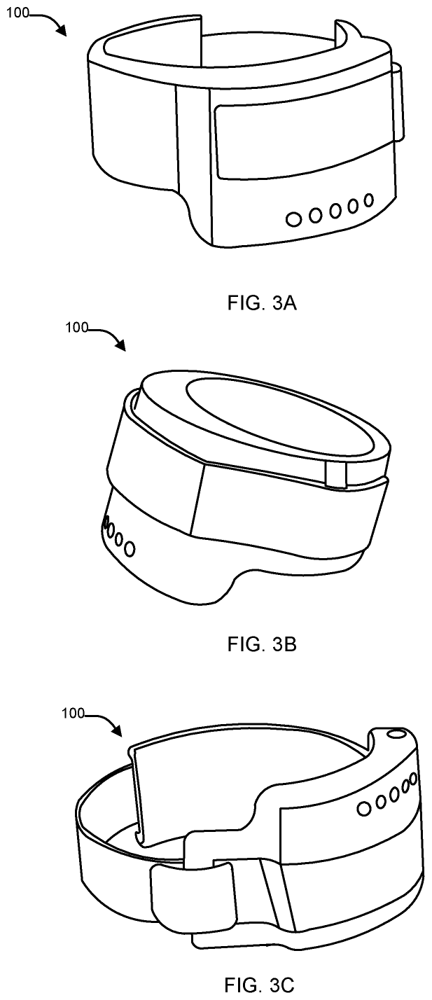

FIGS. 3A-3C illustrate perspective views of the penile device 100 of FIG. 2A, showing therein capability of the penile device 100 to be adjusted to different sizes, such as a large size, shown in FIG. 3A, a small size, shown in FIG. 3B and a medium size, shown in FIG. 3C.

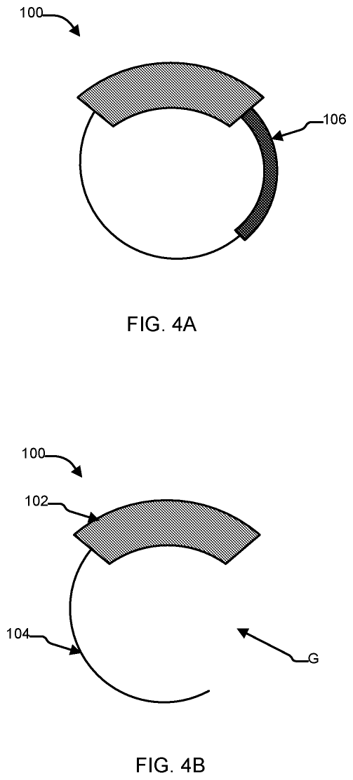

FIGS. 4A-4B illustrate schematic representations of the penile device 100 of FIG. 2A, showing arrangement of the electronics housing 102, the stretch sensor 104 and the gap filler 106 in relation with each other. As shown in FIG. 4B, the one end of the stretch sensor 104 is physically and communicably coupled with one end of the electronics housing 102, the other ends of the stretch sensor 104 and the electronics housing 102 being free with a gap, shown as ‘Cr’, for fixing the gap filler 106 between them, as shown in FIG. 4A, for enabling adjustment in size of the penile device 100.

In an embodiment, the stretch sensor 104 is stretchable in length, and deforms under a force exerted by a penis due to increase in diameter during erection from a flaccid state. Further, the stretch sensor 104 is configured to come back to its original length when the penis comes back to flaccid state. The stretch sensor 104 is configured to monitor and measure the change in length that results from a force exerted on the stretch sensor 104, which happens due to change in circumference of the penis during such change of states. In an embodiment, the stretch sensor 104 may be made of a material that undergoes change in properties due to deformation, and such variation is received by the electronics housing 102 as electrical signals. The stretch sensor 104 effectively communicates the details to the electronics housing 102 in real time.

In an embodiment, the stretch sensor 104 of the penile device 100 can be made of a electroactive polymer to enable its use both as a stretch/strain sensor as well as to alter the applied force and thus can be used for measuring tumescence as well as rigidity.

In an embodiment, to cater to different sizes of penis in flaccid state, the gap filler 106 is adjustable in size. In an example, size associated with the gap filler 106 may be adjusted manually when the penile device 100 is being disposed on the penis. The adjustable feature of the gap filler 106 enables the penile device 100 to be put snugly, close to the penile shaft, so that as the penis undergoes erection, the penile device 100 tends to expand, resulting in increase in length of the stretch sensor 104 to enable detection of the erecting state of the penis.

In an embodiment, the penile device can further include means for accurate determination of the penile circumference in absolute values. The means can be implemented by identifying exact length of the adjustable gap-filler. This may be achieved, for instance, using a linear encoder integrated with the gap filler. The linear encoder can be, but not limited to, capacitive encoder, an optical encoder, an inductive or magnetic encoder. In this aspect, the electronics housing can have an electronic reader for the encoder. Combined with the length of the dynamic stretch sensor and the known length of the electronics housing, the signal from this linear encoder can be used for accurate determination of the penile circumference in absolute values.

In an embodiment, the sleep characteristic module 214 is configured with the head-band device 300, wherein the head-band device 300 is implemented as a sleeping mask with electrodes strategically placed around the eyes for EOG measurements. In an alternate implementation, the electrodes on the forehead could be used to collect EEG data, instead of EOG data, to track sleep and determine sleep-state. Furthermore, the sleep phases may be determined by additional sensors or a physical system working independent to the disclosed system, such as an infrared video system.

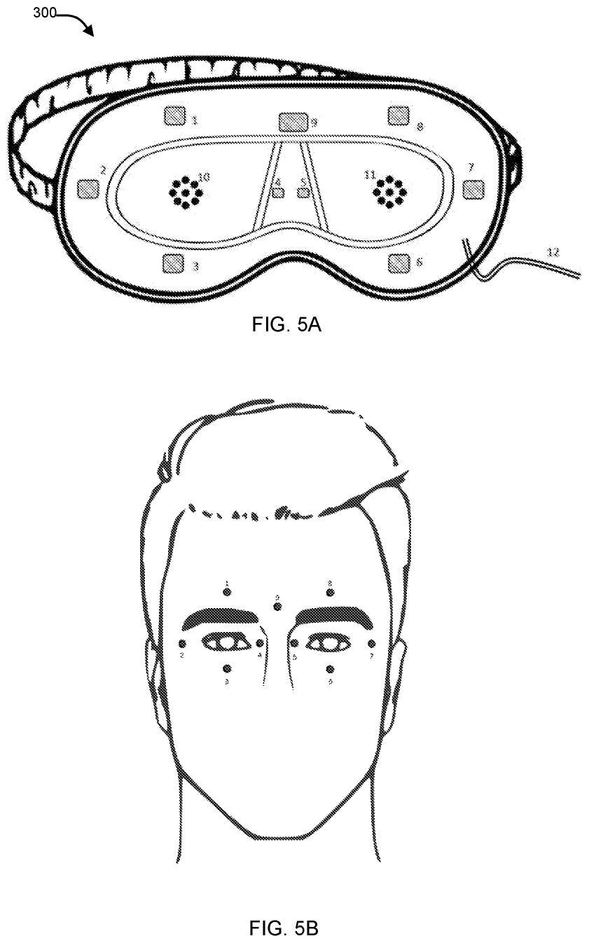

FIG. 5A illustrates an exemplary head-band device 300 incorporating the sleep characteristic module 214. FIG. 5B illustrates schematic of face of a subject or patient for wearing the head-band device 300. As shown in FIGS. 5A-B, the head-band device 300 includes a plurality of electrodes 1-9 configured to correspondingly contact the facial points 1-9, upon wearing of the head-band device 300 by the subject or the patient. The head-band device 300 also includes perforations 10-11 in the sleeping mask which give the patient the ability to see-through the mask. The head-band device 300 also includes a wire 12 connecting the sensor-sets. In an example, the head-band device 300 may include a battery and other communication electronics to enable receiving of data from the sensor-set wirelessly through Bluetooth® LE. Alternatively, the head-band device 300 can use Zigbee® or Wi-Fi® communication protocols. It can also have a wired link using I2C® bus or SPI® or USB® as communication protocols.

In a normal use-case scenario, a patient or subject places the penile device 100 around the penile shaft before going to bed and keep them on overnight. In an example, the system 200 can also detect and proper placement of the penile device 100 and the head-band device 300 by using signals received from the sensors. Once the proper placement of the sensors is verified the data-acquisition process begins and the pre-processed data is stored locally in an EEPROM or the server 400.

In an implementation, the system 200 may also be used without active communication of the penile device 100 and the head-band device 300 with the computing device. A small display incorporated in the head-band device 300 can provide user real-time feedback on the status of the device. A simple feedback system can be realized by placing a small multi-color LED towards the inner side of the head-band device 300. The LED provides visual feedback to the user during operation of the device 300 for easier adjustment.

For instance, no light from the multi-color LED can indicate that all sensors are placed properly and a successful data-acquisition is expected. On the other hand, lights of different colors from the LED can indicate different situations. For example, blue light can be associated with improper placement of the EOG/EEG sensor, red light can be associated with improper placement of the penile device, green light can be associated with improper placement of the system 200 running in debug mode for troubleshooting and the required diagnosis cannot be performed. Likewise, yellow light can indicate that the data memory is full or shall be filled in the middle of the overnight operation. A flashing red-light can be associated with occurrence of a device-fault, and the device cannot be used for further diagnosis. In each case, the patient can take appropriate action to remedy the fault.

In an exemplary embodiment, a small vibrating motor or element can be included to warn and wake up the user in case a critical issue arises. For example, the user or subject can be alerted and waken up in case the system determines that the penile tumescence is very high and the penile device 100 may cause reversible or irreversible tissue damages.

Further, in an embodiment, the sensory data processing module 216 is configured to receive and process collected data from the penile device 100 and the head-band device 300. For example, in the morning, the patient or subject can synchronize the penile device 100 and the head-band device 300 to a smartphone or a computer device, on which the system 200 is implemented, to transfer the stored data. The data stored in the EEPROM is read and sent over to the patient's smartphone by using Bluetooth Low-Energy (BLE®) communication protocol. The data is received by a dedicated application running on the smartphone. The data is gathered over few nights (usually three), and the processing of the data is performed by the application alone or together with the remote internet server 400 to complete the diagnosis.

In an example, the sensory data processing module 216 utilizes heart rate for determining sleep-state, because heart-rate is lower during sleep than when the user/patient is awake. Heart-rate can be determined by processing EOG/EEG user's data. Heart-rate can be also determined by processing signals from the tumescence sensor and electro bio-impedance sensor. Heart-pulsation generates burst of blood flow, which directly affects the electro bio-impedance observed in the bio-impedance data. The pulsation frequency corresponds to the heart rate and can be determined by processing this bio-impedance data either digitally or by using a dedicated electronic circuit.

Similarly, heart-pulsation creates dimensional variations of blood vessels and organs which, in turn, affects the radial circumference of the penile-shaft. These variations are more noticeable when the penis is erect than when it is flaccid. The tumescence and rigidity sensor can detect these variations, which appear as small-amplitude periodic variations in signals. These signals are further processed digitally or using a dedicated electronic circuit to determine the user's heart-rate. Furthermore, amplitude of these variations is a good indicator of the rigidity of the penile-shaft and can be used as a primary data-source for determining rigidity or as a secondary data-source to validate rigidity values obtained by other sensors.

Apart from determination of heart-rate, the heart-pulsation signal detected by the tumescence sensor and electro bio-impedance sensor is a characteristic signature of the user and can be used to ascertain his identity.

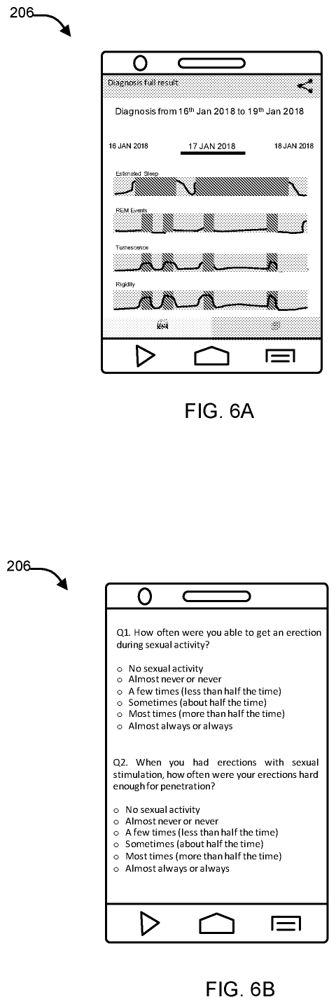

Further, in an embodiment, the recommendation module 218 is configured for an interaction with the subject or user or patient. The system 200 uses a unique approach to interact with its users. In the preferred embodiment, an app running on a smartphone acts as the primary interface 206. In an example, the system 200 provides a graphical representation of various evaluated parameters associated with a user. For example, FIG. 6A illustrates full result of diagnosis wherein various parameters like estimated sleep, REM events, Tumescence, Rigidity, etc. are graphically represented to the user. Further in another example, a user may undertake a standard questionnaire either through this app or under the supervision of a medical practitioner.

FIG. 6B illustrate different questionnaire provided by the system 200 to the user/subject/patient in accordance with an embodiment of the present disclosure. Based on the score, evaluated by the system 200, of the questionnaire test, the use of the proposed device 100 or the system 200 may be prescribed to the user. To reduce misuse or unnecessary use of the device 100, the user who purchases the device online will be asked the unique code generated at the end of the test on the app. The user performs additional tests with the device as per the directions highlighted in the previous sections. Once the diagnosis is completed, the results are displayed on the app for further actions. Furthermore, the data and the result of the diagnosis could be encrypted and stored locally (or in a cloud storage) or in the server 400 for future retrieval and reference.

Further, in an embodiment, the recommendation module 218 provides the patient with post-diagnosis assistance and action-steps based on the outcome of the diagnoses. Some of the possible assistance options are: facilitating the ED patient's online/offline medical consultation with a nearby urologist or psychotherapist; self-help courses; a qualified medical practitioner for the prescription of ED drugs; alternative medicines, etc.

Further, in an embodiment, the machine learning module 220 of the system 200 enables differential diagnosis with higher accuracy by accounting for several biological and racial variations, and evaluates phenotypical variation in penile shape and sizes. In an example, the machine learning module 220 may be configured with a neural network-based hardware module or circuitry of the penile device 100 and the head-band device 300. The machine learning module 220 may be based upon an artificial intelligence (AI) and is configured to capture the phenotypical variations when trained with the relevant data, and characteristics such as ethnicity, height, body-type etc. to provide a holistic score and differential diagnosis of erectile dysfunction.

Further, the machine learning module 220 is configured to predict the root-cause of organic erectile dysfunction. Real-time variations in circumference and rigidity registered by the penile device 100 placed at the base and head of the penile shaft can be used to predict blood-flow dynamics. This evaluation may provide the necessary information for likely root-cause of the problem within a short time and brings in tremendous time-saving in overall treatment of Erectile Dysfunction. This predictive analysis, by the system 200, can be either done by using mechanical penile characteristics such as shape and sizes, or even with or without electro-bio impedance data captured through the electrodes of the head-band device 300.

The system 200 (i.e. system to act as a user interface for data and results visualization) as described in the aforementioned description may be implemented in a smartphone running the system 200 app, which is represented in FIGS. 6A-B. It can also be a standalone computational device such as a personal computer. Alternatively, it can be physically incorporated in the penile device 100 and the head-band device 300. Upon successful connection, the system 200 receives the data from the device 100, 300 and displays it after further processing in the user interface 206, which the patient or the doctor can easily understand. The user can view, manipulate, export and share the data with other parties (including a doctor) for further actions.

In an exemplary embodiment, processing of the data gathered by the system 200 may be done at the server 400 reachable via internet. However, it can be either a part of the head-band device 300 or a local machine running on a remote computation machine on a local area network (LAN) or wide area network (WAN). The server 400 can be even physically incorporated together with the system 200 to facilitate follow-up assistance based on the patient's results and preferred choices. The selected choice decides the course of treatment or action. This system 200 includes one or more front-end UI 206 and back-end applications. The front-end application is an application running on a computational device such as an Android smartphone or a Window® computer. The back-end application can be an application running on a Linux® server system. Depending upon the user's choice, one or more of the following actions can be triggered—
The data is uploaded from the device to a local or remote server 400, where it is shared with a desired doctor or clinician or is retrieved back by the user for future reference. The patient may choose to pay a small fee for the consultation and results to be reviewed by a remote urologist.
The patient is referred to the nearest urologist or psychotherapist for follow-up treatment. The appointments and recordkeeping are managed by the app and the back-end server 400.
The patient chooses to select one or more self-help programs (including subscription to alternative medicine and/or subscription medicine, self-help online/offline courses for life-style changes).
In case the data on tumescence and rigidity date suggests the possibility of hypogonadism, the patient is advised to consult either an endocrinologist or healthcare clinics specialized in low-testosterone diagnosis and care.

Continuous data-logging capabilities of the penile device 100, and in turn the system 200, provides additional dynamic information compared to conventional systems which may only capture data periodically. The captured data can be put through a digital signal processor (DSP) or a similar data-processing system to identify subtle variations registered by the sensors (notably using Fourier transformation of the signal). These variations can be used for determination of maladies which were impossible to be detected by the conventional systems.

In alternate embodiments, apart from addressing erectile dysfunction cases, the system 200 can be modified or used in the current form to detect sleep-issues or other underlying health issues including cardiovascular problems.

The following description provides various structural and functional aspects of the penile device 100 in accordance with different embodiments of the present disclosure.

As mentioned earlier, the penile device 100 is configured to diagnose, monitor, and measure penile tumescence, rigidity, and bio-impedance of penile shaft of the penis.

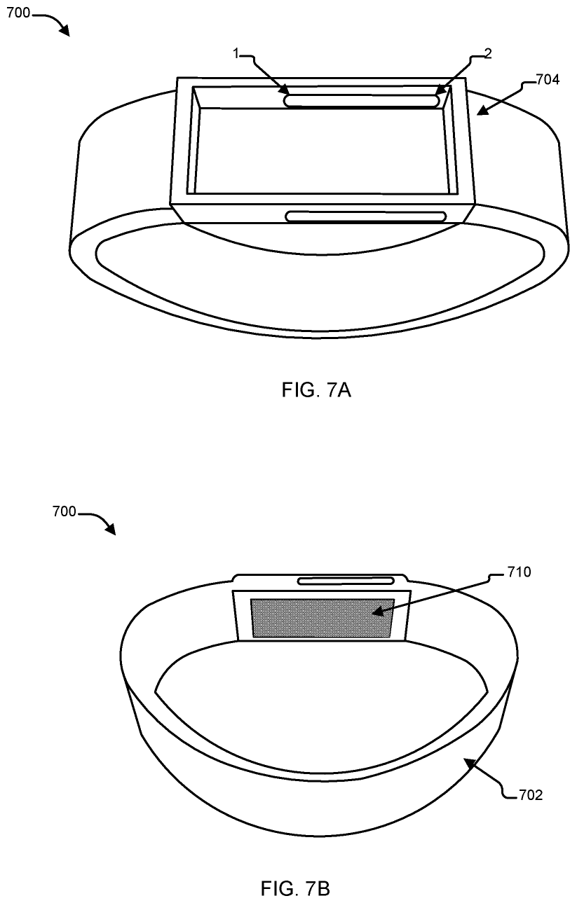

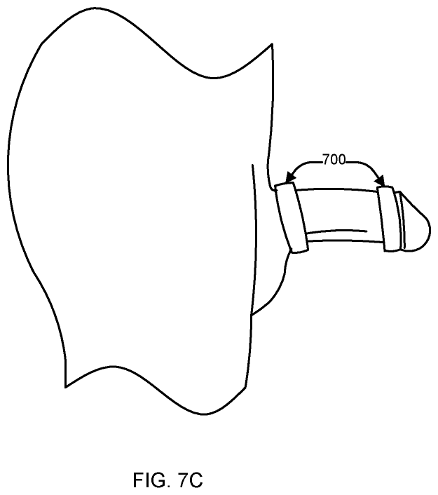

FIGS. 7A-B illustrate different perspective views of a penile device 700 in accordance with another embodiment of the present disclosure. In this embodiment, the penile device 700 is provided as a ring type band having an elastomeric band 702 and an elastomeric housing 704.

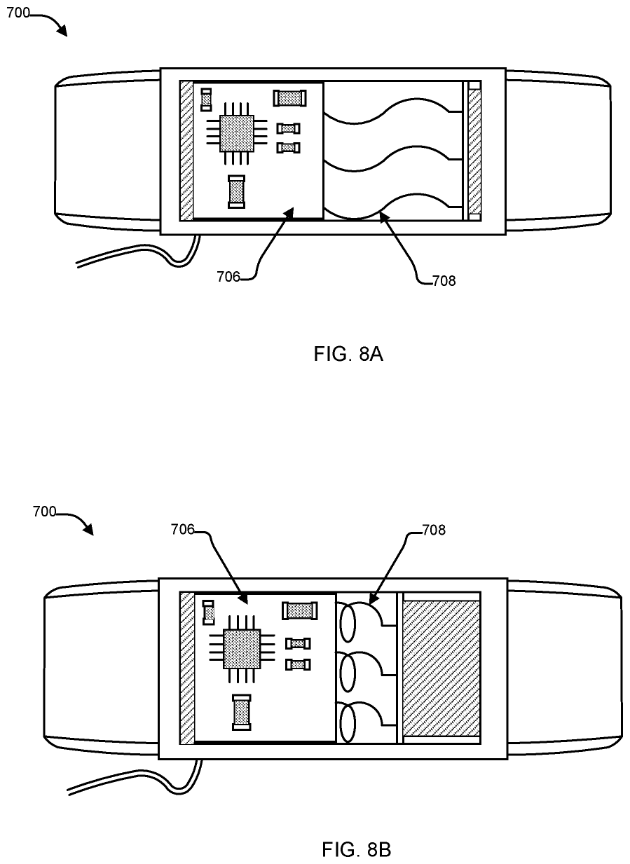

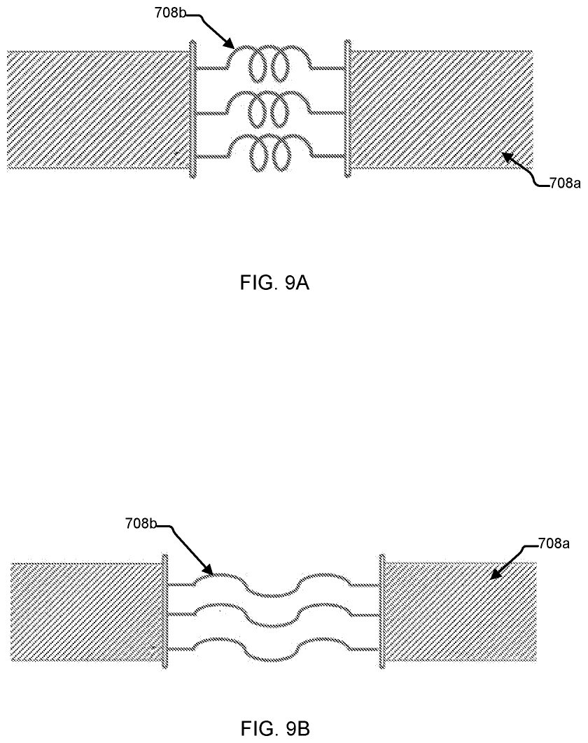

In an example, as shown in FIG. 7C, two penile devices 700 may be accommodated on a penile shaft. The system 200 uses two physically separate penile devices 700. Each device 700 contains sensors to measure tumescence, rigidity and electro-bio-impedance of the patient's penile shaft. Notably, each device 700 (FIGS. 8A-B) consists of electronic circuitry 706 to measure bio-impedance and a sensor 708 which is capable of measuring both tumescence and rigidity. This sensor 708 is developed by combining a first element 708a, provided in the elastomeric band 702, capable of measuring strain (or stretch) with a second element 708b, provided in the elastomeric housing 704, able to perform linear actuation in a small-form factor system (FIGS. 9A-B). In an example, the second element 708b is based on shape memory alloys (SMA). The elements 708a, 708b are joined together end-to-end to form a closed-loop mechanical ring which needs to be placed around the patient's penis. Additional sensors or mechanical elements can be added in this ring. The ring can also be opened and held around the penile shaft by adhesives or another mechanical part.

In another embodiment, the first element 708a may be a capacitive strain (stretch) sensor element combined with a linear actuator such as a shape memory alloys (SMA) to form a ring. Other technologies including resistive strain sensors can be used instead of the capacitive sensors. The elements are arranged together in the elastomeric housing 704 similar to FIGS. 8A-8B. Referring to FIG. 7A, the position 1 and 2 indicate movement limits of the linear actuator or the second element 708b during operations. FIGS. 9A-B represents how the two elements 708a, 708b are combined and show their different working states, i.e. SMA actuator (second element 708b) contracted and expanded. As shown, the SMA actuator (second element 708b) includes a plurality of micro-springs. Accordingly, FIG. 9A correspond to tumescence measurement mode, and FIG. 9B correspond to rigidity measurement mode.

When the penile device 700 is placed around the penile shaft, the linear actuator or the second element 708b, represented in FIGS. 9A-9B by three SMA micro-springs, expands (deforms) to fit snugly on the patient's penile shaft. The stretch sensor or the first element 708a reading contribute to determining whether the penile device 700 is too tight to cause discomfort (or even tissue damage) in the patient or too loose to affect measurement results. This information is used to suggest the patient to adjust the penile device 700 with a smaller or larger circumference.

Further, the system 200 is configured to measure rigidity of the penile shaft at any time on application of electric current across the actuators. For example, once the system 200 has ensured that the patient is in REM sleep phase (through the head-band device 300) and a desired level of tumescence has been reached, a known electric current is applied across the SMA elements (actuator) of the second element 708b. The applied current can be A.C., D.C. or the combination of both (pulsed).

This change in shape reduces the circumference of the penile device 700, thereby providing information from which the rigidity of the penile shaft can be deduced. If the rigidity is high, the stretch sensor 708a will register a higher change in value when the SMA actuator 708b contracts as the rigid penile tissues will resist radial forces and circumferential change will have to be compensated by stretching of the stretch sensor 708a. Similarly, if the rigidity is poor, the stretch sensor 708a will register smaller change in length.

At a given time, two penile devices 700 are used around the penile shaft (FIG. 7C). The first one is placed at the base of the penile shaft and the second one is usually placed close to or at the penile glans. Although one penile device 700 is sufficient to gather tumescence and rigidity data during NPT event, two or more penile devices 700 are required for achieving reliable analysis along with bio-impedance measurements. The tumescence and rigidity provide information on whether the underlying cause of the ED condition is hypogonadism as patients suffering from hypogonadism registers good tumescence but poor rigidity.

Referring back to FIG. 7B, the penile device 700 also includes a bio-impedance sensor 710. Apart from tumescence and rigidity, bio-impedance across the penile shaft provides vital information regarding the blood-flow in the penis. Bio-impedance is measured by applying an electric-potential (A.C., D.C., pulsed, or their combination) across the electrodes of the bio-impedance sensor 710 which are situated underneath the penile device 700. The electric potential can be modulated to reduce environmental noises. The electrodes are in contact with the patient's penile skin and are made of flexible and conductive material to match the penile circumference. Typically, they contact the penis at the base of the penile shaft and the penile glans.

The bio-impedance signal provides a way to monitor blood flowing in and out of the penis through arteries and veins. The signal is used to determine tumescence episodes corresponding to increased blood-flow during a penile tumescence event. In case the erectile dysfunction is of organic type, the obtained bio-impedance data can be further processed to identify the root-cause of the problem. For instance, in case the organic ED is caused by improper blood flow due to clogged arteries or other cardiovascular diseases, the blood inflow will be abnormal and it will be indicated in the temporal bio-impedance measurements. Similarly, if organic ED is due to venous leak, this will be evident by abnormal bio-impedance values.

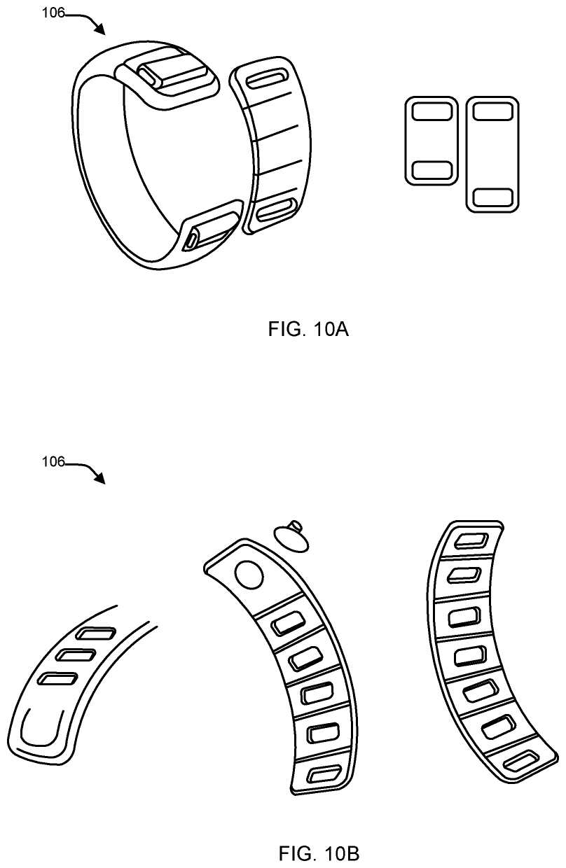

Referring back to FIGS. 4A-4B, the penile device 100 provides the gap filler 106 which is adjustable in size. FIGS. 10A-10B provides two possible implementation of gap filler 106 for accommodating flaccid penis circumferences: (FIG. 10A) by using gap fillers of different given lengths, and (FIG. 10B) by using a band with perforations and a hook mechanism. The gap filler 106 (FIG. 10A) comes in different predefined lengths and connects to the electronic housing 102 via dedicated connectors. The user can choose the appropriate length of the gap filler by selecting one among the gap fillers of different lengths. The gap filler 106 (FIG. 10B) may be trimmed down to the desired length which can then be connected to the electronics housing 102 via dedicated connectors.

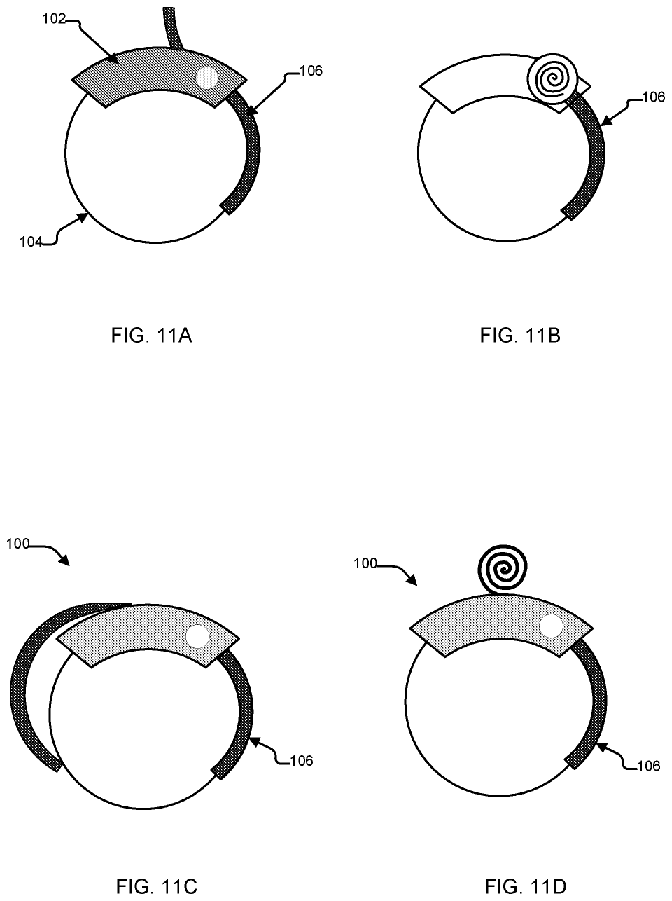

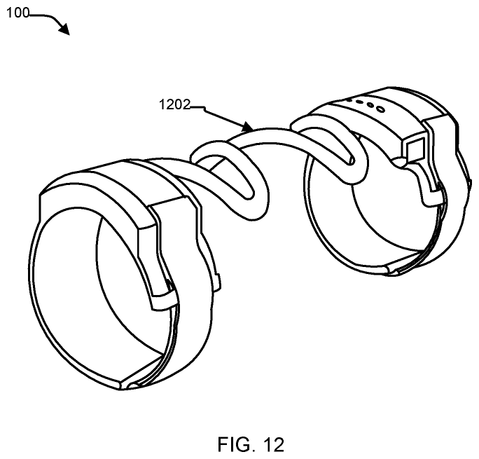

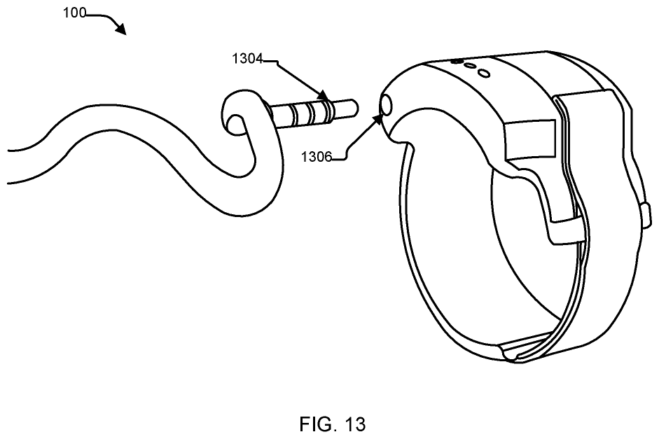

FIGS. 11A-11D illustrate the penile device 100 provided with the gap filler 106 in accordance with different embodiments of the present disclosure, wherein the gap filler 106 is configured to be adjusted in length.

FIG. 11A shows a mechanism where the gap filler 106 can be pulled through an opening in the plastic housing of the electronics housing 102. The physical mechanism may have at least a way to allow user to freely pull the gap filler 106 when desired and engage a mechanism which holds the desired length in place. This mechanism could be a pull or push based mechanical system which causes an interference fit to hold the gap filler 106 in place. This interference fit can be achieved by friction, fastener or even reversible adhesive tape. Fasteners such as hook and loop fasteners are easy to use, low-cost and are very effective in creating an interference fit. In an embodiment, a double-sided hook and loop is used as the gap filler 106 and the corresponding loop fastener is present on the top surface of the electronics housing 102. This loop fastener does not extend over to the elastic part of the electronics housing 102 which ensures that the gap-filler 106 does not interfere with the operation of the strain sensor. The gap filler 106 can even have concurrent hook and loop fastening features so that the excessive length of the gap-filler can be rolled around it.

FIG. 11B provides a mechanism to roll the excessive gap filler 106 along a bobbin. An additional mechanical system freely allows winding of the bobbin in one direction, however, the mechanical system does not allow the gap filler 106 to unwind by itself. The mechanical system requires user's intervention to allow unwinding of the gap filler 106. Thus, holding it in place during the measurement and easily removable when user want to release it. As provided, the gap filler 106 of FIGS. 11A-B allows for smooth length adjustment.

FIG. 11C is an extension of the mechanism of FIG. 11A where excessive gap filler 106 has certain shape-memory which enables it to coil itself around the electronics housing 102 without interfering with the working of the strain (stretch) sensing part of the electronics housing 102. FIG. 11D is an extension of the mechanism of FIGS. 11A and 11C where excessive gap filler 106 has shape-memory to coil around itself to form a small coil. The coil could be gently unwound by handing by applying small force for adjustment of taking it off. FIGS. 11C and 11D illustrate a means of preventing the gap filler 106 “overhang” to interfere with measurements or getting stuck somewhere.

FIG. 12 illustrates another exemplary embodiment implementing the penile device 100 in accordance with an embodiment of the present disclosure. In this embodiment, two penile devices, such as but not limited to penile devices 100, are disposed over the penile shaft, and are coupled to each other through connector 1202. The connector 1202 is able to accommodate changes in separation distance of the two penile devices during an erection from flaccid state, and thereafter return to the flaccid state. The penile devices may be flat or bend to improve fit on the penile shaft. The length-adjustable connectors 1202 can be achieved in one of many forms, examples of which, but not limited to, are springs, motorized screws, electroactive polymers, or linear actuators. In an embodiment, the system 200 also comprises a means of tracking separation distance of the two penile devices 100. This can be achieved through mechanical, electrical or optical means.

FIG. 13 illustrates an exploded view showing coupling of the length-adjustable connectors 1202 with the penile band device 100. As shown, the length-adjustable connectors 1202 may include a male type audio jack 1204 for correspondingly received in female receptacle 1206 provided on the penile band device 100. In an example, the two penile devices 100 may also communicate with each other through the length-adjustable connector 1202. In alternative examples, the length-adjustable connectors 1202 may be coupled with the penile band device 100 through any fastening mechanism.

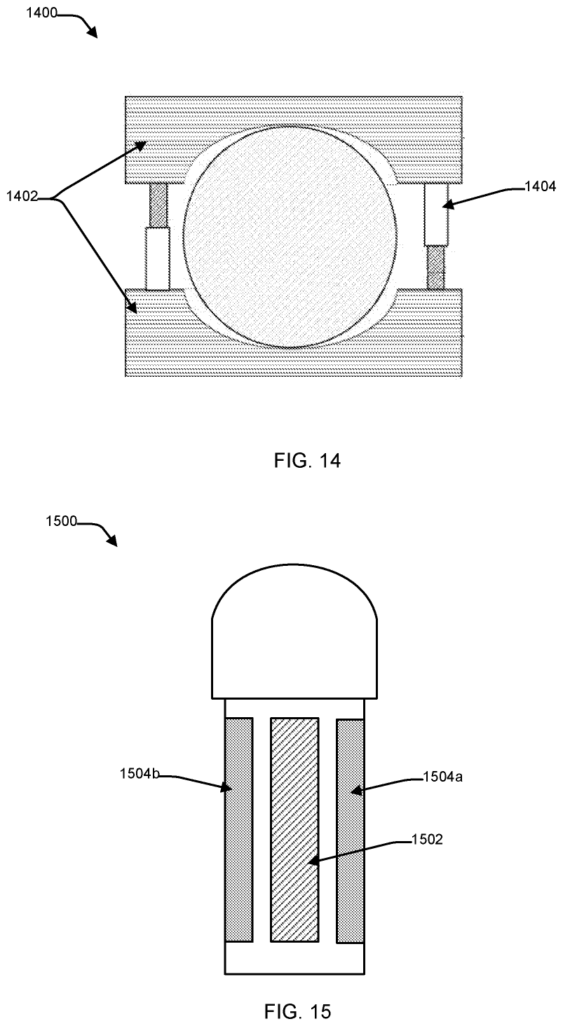

FIG. 14 illustrates another embodiment depicting a penile device 1400 in accordance with an embodiment of the present disclosure. The penile device 100 includes a plurality of clamping plates 1402, along with the length-adjustable connectors 1404, placed on the penile shaft in a manner such that the penis is in between the two plates 1402. The plates 1402 are held in position along the length of the penile shaft through application of a force pushing the plates 1402 together which ensures constant contact of the penile tissue with the two plates 1402. When an erection occurs, the penile circumference will increase and push the plates 1402 further apart. The separation distance between the plates 1402 can be used to approximate the circumference of the penile shaft at any time. For rigidity measurement, the force pushing the plates 1402 together is increased and the change in separation distance is observed. If the penis is rigid, the separation distance will change little upon activation of the extra force. If the penis is flaccid, the change in separation distance between the plates 1402 will be greater.

Instead of using the change in distance between the plates 1402 as indicator of rigidity, the system 200 can also be designed in a manner where a (gradually/continuously) increasing force is applied to push the plates 1402 together until a pre-determined change in separation distance is achieved. The force necessary to achieve this change in separation distance will then indicate rigidity.

In a further embodiment of the invention, the plates 1402 are connected by a hinge on one side and a length-adjustable connector on the other side. In yet a further embodiment of the invention, the plates 1402 are connected via length-adjustable connectors or hinge on one side only and are open on the other side.

FIG. 15 illustrates a penile device 1500 in accordance with another embodiment of the present disclosure. In this embodiment, a new method of penile axial rigidity measurement is provided that comprises at least one stretch sensor straps 1502 and at least two shape memory straps 1504 made of material able to change their shape back to a predetermined shape when subjected to an external activation signal. Examples of such materials, but not limited to, are shape-memory alloys and dielectric electro-active polymers. In an example, a shape-memory strap 1504a has its activated shape at an angle whereas another shape-memory strap 1504b returns to a straight strap when activated. In an example, the shape memory straps 1504a, 1504b are soft actuators.

The shape-memory alloy straps 1504 are placed opposite to one another along the length of the penis using some adhesive. The stretch sensor straps 1502 are placed next to the shape-memory straps 1504 or on top of those. To measure axial rigidity, the shape-memory strap 1504a is activated which will exert a force onto the penis trying to bend it. If the penis is rigid, very little penile bending will occur. If the penis has low axial rigidity, the penis will bend more. The amount of bend will be characterized by the change in stretch detected by the stretch sensors 1502. Depending on the direction of bend and position of the stretch sensor, the stretch sensors 1502 will detect either a reduction in stretch or an increase in stretch. To return the penis back to the original straight position, shape-memory strap 1504b will be activated, which has its activated position in a straight configuration. In another embodiment of the invention, a material combining the stretch sensor 1502 and shape-memory 1504 properties may be used such that only a total of two straps are required. In an alternate example, the penile device 1500 may also be configured to measure both axial and radial rigidity of the penis.

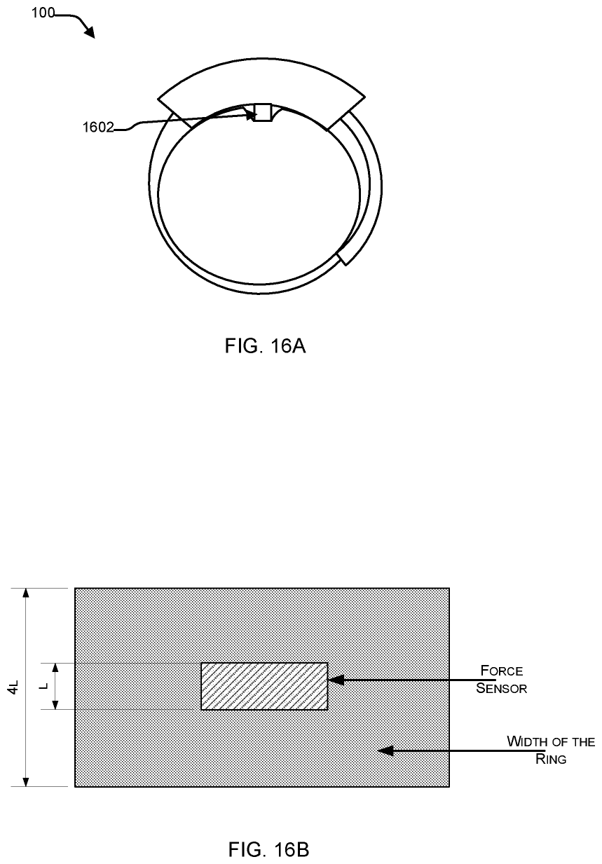

FIG. 16A illustrates the penile device 100 in accordance with another embodiment of the present disclosure. In an embodiment, the penile device 100 includes a force sensor 1602 disposed on the electronics housing 102 and protruding towards the penile shaft. The penile device 100 of this embodiment utilizes the force-sensor 1602 along with the combination of active and passive mechanical elements, to make the penile device 100 suitable for simultaneously deducing circumference of the penis as well as the reactionary force applied by the penile tissue. The sensor 1602 for measuring force is placed in such a way that it measures a radial reactionary force generated by the tissue of the organ on the mechanical elements. In such a system, the force exerted by the tissue will be proportional to the mechanical stiffness (or rigidity) arising due to pressure within the organ. In the simplest form, the mechanical elements can be either elastic, non-elastic or both and are joined together to form a ring.

Using the above approach, the penile device 100 can be configured to measure cavernosal pressure without restricting blood flow or causing discomfort. The pressure due to the hoop stress within the penile device 100 remains lower than the lowest amount of pressure that can affect or hinder the normal blood flow in the penile shaft, or cause discomfort. Likewise, the contact area and the geometry of the force-sensor 1602 for the organ-tissues can be adjusted so that the force exerted by the tissues and arising due to the internal organ pressure onto the contact area of the force-sensor 1602 is always less than the corresponding cylindrical stress force. Pressure (P) exerted on a surface is a function of applied force (F) and the surface area (A), i.e., P=F/A. If the force-sensor 1602 with a given surface area is placed within the penile device 100, it will experience total radially inward force resulting from the outward reactionary force exerted by the penis due to the contraction force in the band of the penile device 100. The reactionary penile force on the force sensor 1602 will be proportional to the penile cavernosal pressure, which in turn will be an indication of penile rigidity.

If the specific surface area of the force sensor 1602 protruding out of the inner surface of the penile device 100 is small, then the same outward reactionary force exerted by the penis will translate into higher applied pressure on the penis. For example, the force experienced by a force sensor 1602, having a smaller surface area, will be many times more than experienced by the ring with a larger surface area (Refer FIG. 16B). Thus, making it possible to measure higher pressure (say 120 mm hg) compared to a lower pressure (say 30 mm hg) applied by the ring on the penile shaft. The lower overall pressure along the ring ensures comfortable use by the patient. Thus, despite having lower hoop pressure in the ring which does not hinder blood flow nor causes discomfort, the penile device 100 with the force sensor 1602 enables system 200 to estimate the cavernosal pressure of the penis. The value of the cavernosal pressure and its periodic variation can be used to score the rigidity.

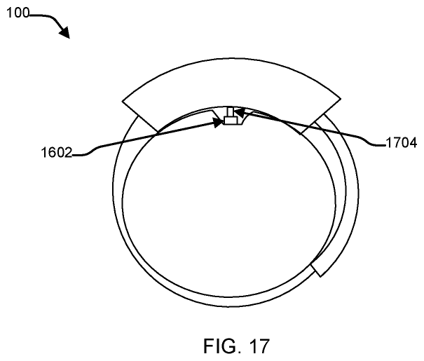

FIG. 17 illustrates the penile device 100 in accordance with another embodiment of the present disclosure. In an embodiment the penile device 100 includes a force sensor 1602 disposed on the electronics housing 102 and protruding towards the penile shaft. In an example, the force sensor 1602 is mounted on a retractable mechanical system 1704 with its closed position flushed with the surface of the ring. The force sensor 1602 can then protrude out for a brief period to take a measurement and then retracts back. The force reading collected during the retracting motion and when the force sensor 1602 protruded to its maximum length can be used to determine the cavernosal pressure and/or rigidity.

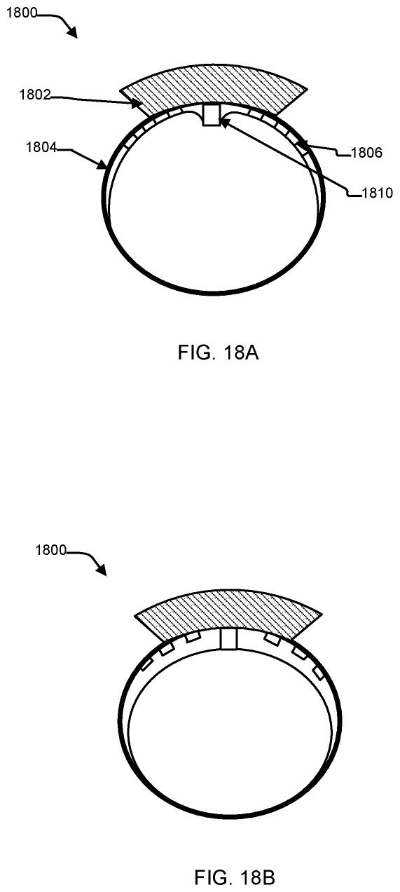

FIGS. 18A and 18B illustrate another penile device 1800 in accordance with an embodiment of the present disclosure. In an embodiment, the penile device 1800 includes an electronics housing 1802 (similar to the electronics housing 102), and a band 1804 (similar to the stretch sensor 104). The band 1804 may be elastic or inelastic. The elastic band 1804 may include several touch sensors 1806 (for instance, capacitive touch sensors) that may be integrated in the elastic band 1804 and are placed to one or both sides of a protruding notch 1810 with increasing distance from the notch 1810. In an example, the protruding notch 1810 may be similar to the force sensor 1602. When the penis is flaccid, the indentation of the penile tissue by the protruding notch 1810 will cause all touch sensors 1806 to detect a signal from the penile skin. When the penis is fully erect, fewer (or none) of the touch sensors 1806 will detect a signal as they are no longer in contact with the penile skin. The number of touch sensors 1806 detecting a signal can be used to gauge the rigidity of the penis.

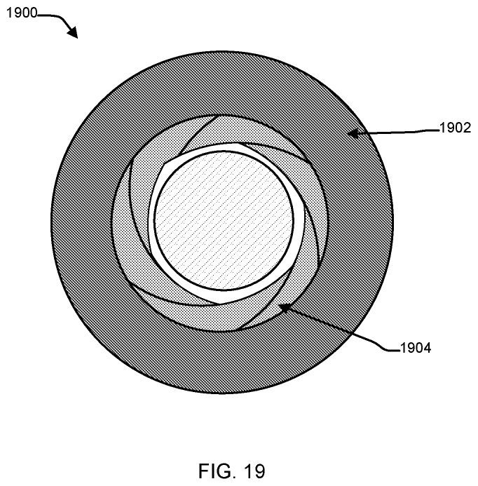

FIG. 19 illustrates a penile device 1900 in accordance with another embodiment of the present disclosure. In this embodiment, a novel approach for tumescence as well as rigidity measurement using an iris diaphragm 1902 is outlined. The iris diaphragm 1902 has adequate number of leaves 1904 (generally more than 5) which creates a quasi-circular aperture opening during its motion. The maximum aperture-opening diameter should be at least equal to the largest possible circumference for the penis. The aperture opening is controlled using a torque which can be generated by myriad of sources such as an electric motor with an ability to vary torque.

The penile device 1900 is placed radially over the penile shaft such that the penis goes through the centre hole 1906. By exerting a constant small torque onto the iris diaphragm 1902 lever, the relative opening of the diaphragm 1902 will always correspond to the penile circumference. If the penile circumference increases, the penile tissue will push the iris diaphragm 1902 further open. The position tracking capability of the iris diaphragm 1902 will detect this change of circumference. If the penile circumference decreases, the constant gentle torque acting on the iris diaphragm 1902 will ensure that the centre hole 1906 of the iris diaphragm 1902 also decreases again. Thus, penile tumescence can be determined following such approach.

The penile device 1900 can be used for determining penile rigidity as well. The torque on the iris diaphragm 1902 will translate as force (pressure) on the penile tissue. By varying the torque, one can tweak the applied pressure on the penile tissue and determine the change in circumference corresponding to an applied torque. By determining the maximum torque needed to cause a change in circumference, rigidity can be determined.

The foregoing description explained different embodiments of the penile device 100, 700, 1400, 1500, 1800, 1900 configured to evaluate one or more of different parameters for diagnosing erectile dysfunction causes. The following table summarizes the parameters evaluated by the penile device 100, 700, 1400, 1500, 1800, and 1900.

| | Parameter | | |
|---|---|---|---|
| Embodiment | Tumescence | Radial rigidity | Axial rigidity |
| Penile Device 100, 700 | YES | YES | No |
| Penile Device 1400 | YES | YES | No |
| Penile Device 1500 | No | No | YES |
| Penile Device 100 | YES | YES | No |
| Penile Device 1900 | YES | YES | No |
| Penile Device 1800 | YES | YES | No |

In another aspect, all the above devices, except the penile device 1500, can also be integrated with a bio-impedance sensor Thus, depending on group of parameters to be monitored and measured; more than one type of penile device can be arranged on the penis to cover the desired range of parameters.

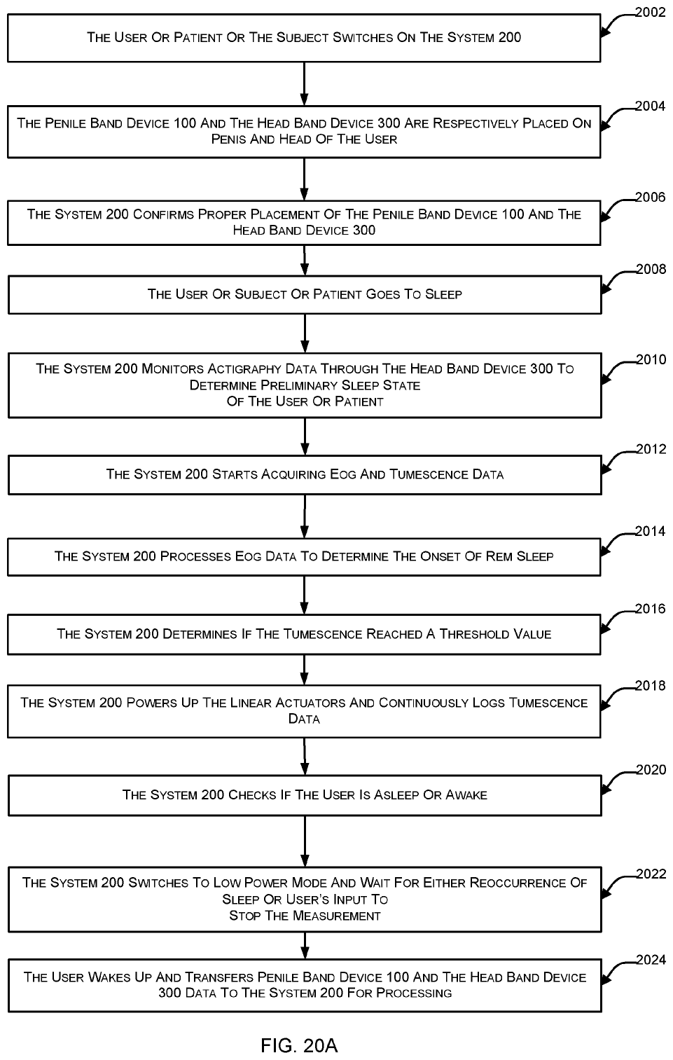

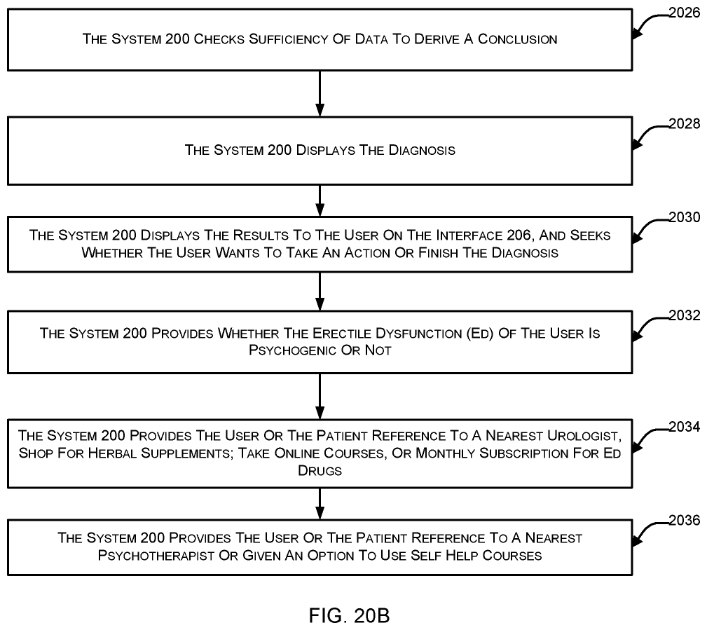

FIG. 20 illustrates a method 2000 for monitoring, diagnosing, and managing a condition of erectile dysfunction associated with a penis in accordance with an embodiment of the present disclosure.

In an embodiment, the method 2000 is implemented through the system 200 configured with the penile device 100 and the head-band device 300 in accordance with an embodiment of the present disclosure. At step 2002 the user or patient or the subject switches on the system 200. At step 2004 of the method 2000, the penile device 100 and the head-band device 300 are respectively placed on penis and head of the user. At step 2006 of the method 2000, the system 200 confirms proper placement of the penile device 100 and the head-band device 300. At step 2008 of the method 2000, the user or subject or patient goes to sleep. At step 2010 of the method 1900, the system 200 monitors actigraphy data through the head-band device 300 to determine preliminary sleep state of the user or patient.

If the user is asleep, at step 2012 of the method 2000, the system 200 starts acquiring EOG and tumescence data. At step 2014 of the method 2000, the system 200 processes EOG data to determine the onset of REM sleep. At step 2016 of the method 2000, the system 200 determines if the tumescence reached a threshold value, for example 80%. In case the tumescence reaches the threshold value, at step 2018 of the method 2000, the system 200 powers up the linear actuators and continuously logs tumescence data. At step 2020 of the method 2000, the system 200 checks if the user is asleep or awake. If the user is awake, at step 2022 of the method 2000, the system 200 switches to low power mode and wait for either reoccurrence of sleep or user's input to stop the measurement.

At step 2024 of the method 2000, the user wakes up and transfers penile device 100 and the head-band device 300 data to the system 200 for processing. At step 2026 of the method 2000, the system 200 checks sufficiency of data to derive a conclusion. If the data received is insufficient, the method 2000 is repeated; else at step 2028 of the method 2000, the system 200 displays the diagnosis. At step 2030 of the method 2000, the system 200 displays the results to the user on the interface 206, and seeks whether the user wants to take an action or finish the diagnosis. It the user opts for an action, at step 2032 of the method 2000, the system 200 provides whether the erectile dysfunction (ED) of the user is psychogenic or not. If the ED is not psychogenic, at step 2034 of the method 2000, the system 200 provides the user or the patient reference to a nearest urologist, shop for herbal supplements; take online courses, or monthly subscription for ED drugs. If the ED is psychogenic, at step 2036 of the method 2000, the system 200 provides the user or the patient reference to a nearest psychotherapist or given an option to use self-help courses.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the spirit of the appended claims.

We claim:

1. A system for monitoring, diagnosing, and managing a condition of erectile dysfunction of a user, the system comprising:
a computing device having a processor and a memory coupled to the processor;
a penile device configured to be accommodated on penis of the user and operatively coupled to the computing device, wherein the penile device is configured to monitor and measure at least one of a penile tumescence, a rigidity of a penile shaft of the penis, and a bio-impedance of the penile shaft; and
a head-band device configured to be accommodated on head of the user and operatively coupled to the computing device, wherein the head-band device is implemented as a sleeping mask with electrodes strategically placed around the eyes of the user for detecting at least one of sleep characteristics, electrooculography (EOG), and electroencephalography (EEG);
wherein the computing device is configured to gather data on at least one of the penile tumescence, the rigidity, and the bio-impedance of the penile shaft of the penis of the user from the penile device, and at least one of the sleep characteristics, the electrooculography (EOG), and the electroencephalography (EEG) of the user from the head device, and to process the gathered data to qualify condition of the erectile dysfunction (ED) of the user.

2. The system as claimed in claim 1, wherein detection of the sleep characteristics include detection of a REM sleep phase; and wherein the computing device, on detection of the REM sleep phase and a condition of penile tumescence, is configured to actuate the penile devise to measure the rigidity of the penile shaft to qualify condition of the erectile dysfunction as organic or inorganic.

3. The system as claimed in claim 1, wherein the head-band device is adapted to determine heart-rate of the user from the electrooculography and the electroencephalography signals, wherein the heart rate is used to determine sleep state of the user.

4. The system as claimed in claim 1, wherein the penile device is configured to be accommodated around the penile shaft, and comprises:
an elastomeric band; and
an elastomeric housing, wherein the housing includes an electronic circuitry to measure bio-impedance through a bio-impedance sensor, and a tumescence and rigidity sensor for measuring both tumescence and rigidity; wherein the tumescence and rigidity sensor includes a first element for measuring strain to detect tumescence, and a second element to perform linear actuation to apply force for measuring a radial rigidity by measuring strain, using the first element, as a result of the applied force.

5. The system as claimed in claim 4, wherein the second element is made of a shape memory alloy and is configured in series with the first element, so as to stretch as a result of tumescence; and wherein the second element regains its original shape on receiving an electric current, wherein the regaining the original shape results in application of radial force on the penile shaft.

6. The system as claimed in claim 4, wherein the penile device is adjustable in size and accommodates a minimum penile circumference corresponding to a flaccid penis, and a maximum penile circumference corresponding to an erect penis.

7. The system as claimed in claim 4, wherein the penile device includes an inward extending force sensor to estimate a cavernosal pressure of the penis shaft, and wherein the cavernosal pressure and its periodic variation is used to estimate the rigidity; and wherein the force sensor has a width narrower than the stretch sensor.

8. The system as claimed in claim 4, wherein the penile device includes a plurality of touch sensors provided on the elastic band on either side of a protruding notch, wherein the touch sensors are configured to provide signals upon being touched and not touched by penile skin based upon respective erect or flaccid state of the penis.

9. The system as claimed in claim 1, wherein the system includes a plurality of penile devices located along length of the penile shaft and coupled to each other through one or more length-adjustable connectors.

10. The system as claimed in claim 1, wherein the penile device is based on a sensor made of a electroactive polymer and arranged around circumference of the penile shaft, wherein the sensor is used to measure strain as a result of change in circumference to measure tumescence, as well as to alter an applied force on the circumference of the penile shaft for measuring rigidity.

11. The system as claimed in claim 1, wherein the penile device comprises two clamping plates placed on the penile shaft in a manner such that the penis is in between the two plates, and wherein the two plates are coupled to each other by at least one length-adjustable connector.

12. The system as claimed in claim 1, wherein the penile device is configured to measure an axial rigidity of the penis and comprises at least one stretch sensor strap and at least two shape memory straps made of material able to change their shape back to a predetermined shape when subjected to an external activation signal, the at least one stretch sensor strap and the at least two shape memory straps are configured along length of the penile shaft; and wherein one of the at least two shape-memory straps has its activated shape at an angle, whereas the other of the at least two shape-memory straps returns to a straight strap when activated.

13. The system as claimed in claim 1, wherein the penile device includes an iris diaphragm having number of leaves forming a quasi-circular aperture opening of variable size and resting against the penile shaft with a constant gentle torque acting on the iris diaphragm, the penile device having position tracking capability of the iris diaphragm to detect change of circumference of the penile shaft to detect tumescence.

14. The system as claimed in claim 13, wherein the iris diaphragm is configured to determine radial rigidity by varying the torque and determine the change in the circumference of the penile shaft corresponding to an applied torque.

15. The system as claimed in claim 1 includes an artificial intelligence based machine learning module that enables differential diagnosis with higher accuracy by accounting for biological and racial variations.

16. The system as claimed in claim 1 the system comprises an interface configured to provide the user any or a combination of a graphical representation of various evaluated parameters, and a questionnaire test, wherein before being accessible to the user, the system confirms authenticity of the user based upon a unique code generated at the end of a test conducted on the user.

17. A penile device configured to be accommodated circumferentially around a penile shaft of a penis, the device comprising:
an electronics housing;
stretch sensor with one end of the stretch sensor physically and communicably coupled with the electronics housing; and
a gap filler fastened between the electronics housing and the other end of the stretch sensor such that the electronics housing, the stretch sensor, and the gap filler take a flexible band shape for being accommodated on the penile shaft of the penis, wherein the gap filler is of adjustable length to enable snug fitment of the band shaped penile device circumferentially around the penile shaft in a flaccid state of the penis;
wherein, the stretch sensor being stretchable stretches under a force exerted by the penis during erection from a flaccid state, and returns to its original shape when the penis returns to the flaccid state; and
wherein the stretch sensor, on being stretched, is adapted to provide a signal indicative of force exerted on the stretch sensor to detect and measure a penile tumescence.

18. The penile device as claimed in claim 17, wherein the stretch sensor is physically coupled to the electronics housing through a linear actuator adapted to contract to reduce circumferential length of the penile device to exert a radial force on the penile shaft to measure axial rigidity of the penile shaft, the measurement of the axial rigidity being based on measurement of force exerted on the stretch sensor as a result of reduction in circumferential length of the penile device.

19. The penile device as claimed in claim 18, wherein the linear actuator is one or more of micro springs made of a shape memory alloy, and the micro springs configured between the stretch sensor and the electronics housing such that the micro springs stretch along with stretching of the stretch sensor during erection of the penis, or before, during arranging the penile device around the penile shaft, and wherein the micro springs return to their original shape on application of a current to reduce circumferential length of the penile device.

20. The penile device as claimed in claim 17, comprising a force sensor disposed on the electronics housing, protruding towards the penile shaft to estimate a cavernosal pressure of the penis shaft, the cavernosal pressure and its periodic variation being used to estimate the rigidity; and wherein the force sensor has a width narrower than the stretch sensor.

21. The penile device as claimed in claim 20, wherein the force sensor is mounted on a retractable mechanical system.

22. The penile device as claimed in claim 17, wherein the gap filler includes a linear encoder, and the electronics housing includes an electronic reader for the encoder; and wherein, based on known dynamic length of the stretch sensor and known length of the electronics housing, the signal from this linear encoder allows determination of the penile circumference in absolute values.

23. A method for monitoring, diagnosing, and managing a condition of erectile dysfunction of a user, the method comprising the steps of:
monitoring, using a penile device accommodated on penile shaft of penis of the user, at least one of a penile tumescence, a rigidity of a penile shaft of the penis, and a bio-impedance of the penile shaft; and
monitoring, using a head-band device configured on head of the user, at least one of sleep characteristics, electrooculography (EOG), and electroencephalography (EEG);
detecting, based on monitoring of at least one of the sleep characteristics, the electrooculography (EOG), and the electroencephalography (EEG), if the user is in an REM state;
detecting, when the user is in an REM state, based on monitoring of the penile tumescence, if the user in a condition of penile tumescence;
actuating, using a computing device operatively coupled to the penile device and the head-band device, when the user is in a condition of penile tumescence, the penile devise to measure the rigidity of the penile shaft; and
qualifying, based on the measured rigidity of the penile shaft, condition of the erectile dysfunction as organic or inorganic.
